SEQUENCE LISTING (9 sequences)

SEQ ID NO: 1 - DNA, 20 bp, artificial sequence
  primer for SHP1
cgaggtgtccacggtagctt

SEQ ID NO: 2 - DNA, 24 bp, artificial sequence
  primer for SHP1 (reverse)
cccctccatacaggtcatagaaat

SEQ ID NO: 3 - DNA, 28 bp, artificial sequence
  probe for SHP1
tgacccatattcggatccagaactcagg

SEQ ID NO: 4 - DNA, 19 bp, artificial sequence
  probe for SHP2 (forward)
gcgacaactgcacggatct

SEQ ID NO: 5 - DNA, 19 bp, artificial sequence
  probe for SHP2 (reverse)
cagcgtcacagcccctaag

SEQ ID NO: 6 - DNA, 23 bp, artificial sequence
  probe for SHP2
ctcgcactgggaatcccctccat

SEQ ID NO: 7 - DNA, 31 bp, artificial sequence
  probe for ABL (forward)
tggagataacactctaagcataactaaaggt

SEQ ID NO: 8 - DNA, 21 bp, artificial sequence
  probe for ABL (reverse)
gatgtagttgcttgggaccca

SEQ ID NO: 9 - DNA, 28 bp, artificial sequence
  probe for ABL
ccatttttggtttgggcttcacaccatt

(19) United States
(12) Patent Application Publication
(10) Pub. No.: US 2011/0312968 A1
(43) Pub. Date: Dec. 22, 2011

(54) METHOD FOR OPTIMIZING THE TREATMENT OF CHRONIC MYELOID LEUKEMIA WITH ABL TYROSINE KINASE INHIBITORS

(76) Inventors: Nicola Esposito, Nopoli (IT); Barbara Izzo, Pozzuoli (IT); Thea Kalebic, Springfield, NJ (US); Fabrizio Pane, Napoli (IT); Fabrizio Quarantelli, Napoli (IT)

(21) Appl. No.: 13/126,683

(22) PCT Filed: Nov. 5, 2009
(86) PCT No.: PCT/US09/63349

§ 371 Date: Jul. 20, 2011

Related U.S. Application Data
(60) Provisional application No. 61/112,221, filed on Nov. 7, 2008.

(51) Int. Cl. A61K 31/506; A61P 35/02; C12Q 1/68

(52) U.S. Cl. 514/252.18; 435/6.11

(57) ABSTRACT

The present invention relates to a method tor evaluating patterns to help-optimizing the treatment of chronic myeloid leukemia (CML) in a human patient population. Specifically, present invention relates to SHP1 and/or SHP2 as a biomarker for CML patients.

The present invention relates to a method of treating chronic myeloid leukemia (CML) in a human patient population.

The success of treatment with Imatinib mesylate in the majority of chronic phase CML patients is well documented. Improving treatment outcomes for those patients who perform less well however requires a detailed understanding of the critical determinants of treatment response.

SHP-1 and SHP-2 are two Src homology 2 (SH2) domain-containing tyrosine phosphatases with several pathological implications on cell growth regulating signaling. They share significant overall sequence identity. Their biological functions are not well elucidated. SHP-1 is generally considered as a negative signal transducer and SHP-2 as a positive one. SHP-2 has been found widely expressed, while SHP-1 is highly expressed in hematopoietic ceils and, at a lower level, in some nonhematopoietic cells.

Both SHP-1 and SHP-2 are thought to have important pathological implications. Namely, SHP-1 dephosphorylates receptors of growth factors, cytokines, and antigens, and tyrosine-phosphorylated proteins associated with these receptors. Therefore, if is often defined as a negative signal transducer. In humans, reduction of SHP-1 gene expression is observed in natural killer cell lymphomas as well as other types of lymphomas/leukemias. Methylation of the SHP-1 promoter causes loss of SHP-1 expression in malignant T-lymphoma cells. Decreased expression level of SHP-1 has been found associated with progression of chronic myeloid leukemia (CML); Moreover, Shp1 was shown to be physically associated with Bcr-Abl which suggests their functional interaction. Furthermore, overexpression of Shp1 blocks transformation by Bcr-Abl.

Activation mutation of SHP-2 causes Noonan syndrome, an autosomal dominant disorder characterized by dysmorphic facial features, proportionate short stature, and heart disease (most commonly pulmonic stenosis and hypertrophic cardiomyopathy). This gain-of-function mutation of SHP-2 is also associated with sporadic juvenile myelomonocytic leukemia, myelodysplasic syndrome, acute lymphoblastic leukemia, and acute myelogenous leukemia. SHP-2 has been described as an intracellular target of Helicobacter pylori CagA protein which is associated with gastritis and gastric cancer. Functional knock-out of the Shp-2 gene in the mouse causes death of embryos at mid-gestation. Cells expressing a catalytically inactive cysteine-to-serine mutant of SHP-2 and those derived from SHP-2 knock-out mice exhibited reduced activation of signal transduction pathways induced by growth factors and cytokine. SHP-2 also has a role in angiotensin II signaling that may be responsible for the defects in heart development associated with its mutation.

It has now been found that two SHP-constitutive non receptor protein tyrosine phosphatases, SHP-1 and SHP-2, play a role in the negative regulation of Ber-Abl and that lack of Shp1may be important for CML transformation.

It is hence an object of the present invention to identify novel prognostic indicators to improve both an initial assessment and subsequent monitoring of CML patients. It is a further object of present invention to specify a patient population for the treatment of CML, in particular by estimating treatment response. It is a further object of present invention to improve success of treatment of CML. It is a further object of present invention to predict achievement of major molecular response (MMR) in CML patients.

Surprisingly, it has been found that not only phosphokinases, but also the phosphatases SHP1 and SHP2 may serve as biomarkers.

Hence, in one aspect, the present invention pertains to the use of SHP1 and/or SHP2 as a biomarker for CML patients. Preferably, the invention relates to the use of SHP1 as a bio-marker for CML patients. Thereby, the level of SHP1 and/or SHP2 is indicative for the therapeutic efficacy of imatinib or a pharmaceutically acceptable salt thereof.

### Definitions:

“SHP1 level”, as used herein, is defined as relative to the level of Abl. “SHP2level”, as used herein, is defined as relative to the level of Abl. Meant is the mRNA levels of SHP1 and SHP2, respectively, assayed by Q-PCR and expressed as ratio to ABL.

It may be stated that measurement of SHP1 level and SHP2 level, respectively, can for instance be carried out on samples taken from bone marrow or blood, preferably of peripheral blood. However, for clarification of the definition, SHP1 level and SHP2 level, respectively, are preferably measured from samples of peripheral blood. The method of determine the level is described below.

“Sample” means blood or bone marrow sample, preferably peripheral blood sample.

The word “about”, as used herein and throughout the application, refers to a value that can vary within a range from of −10% to +10% of the indicated value. Preferably from −5% to +5% of the indicated value.

The term “warm-blooded animal” preferably means a human or human patient. “Patient” preferably relates to a human patient.

The term “Ima” as used herein is synonymous to imatinib or a pharmaceutically acceptable salt thereof, preferably the mesylate salt.

The level of SHP1 and/or SHP2 in a CML patient can be used for the assessment of the therapeutic amount of imatinib or pharmaceutically acceptable salt thereof, as well as for the additive or substitutive treatment of said patient with nilotinib and/or dasatinib or a pharmaceutically acceptable salt thereof. In particular, a level of SHP1 lower than 3 is indicative for raising the therapeutic amount of imatinib or a pharmaceutically acceptable salt thereof, preferably to at least 150% of the standard dosage prescribed for CML patients. Treatment with nilotinib or dasatinib or a pharmaceutically acceptable salt thereof may occur additionally or in substitution of imatinib.

In one embodiment of present invention, the low SHP1 level is lower than 3. in further embodiments, the SHP1 level is from 0.01 to 3. (n further embodiments, the upper limit of the SHP1 level is 3, 2.8, 2.6, 2.4, 2.2 and 2; and the lower limit of the SHP1 level is 0.01 or 0.1. It is understood that all combinations of upper and lower limit are comprised by present invention.

Hence, in one aspect the present invention pertains the use of SHP1 and/or SHP2 as a bio-marker for CML patients for determining the therapeutic efficacy of imatinib or a pharmaceutically acceptable salt thereof.

In a further aspect, present invention relates to an ex vivo method for determining the SHP1 and/or SHP2 level, comprising the steps of
a) determining the mRNA level of SHP1 and/or SHP2 from a sample;
b) determining the mRNA level of ABL;
c) normalizing SHP1 and/or SHP2 mRNA to ABL.

Preferably, SHP1 level is determined with such ex vivo method. Determination and normalizing is preferably performed with the methods as described in the experimental section below. Preferably, the blood sample is a peripheral blood sample.

A further aspect of present invention relates to the use of the above ex vivo method for screening CML patients to determine appropriate treatment with imatinib, nilotinib, and/or dasatinib, or a pharmaceutically acceptable salt thereof. The term “appropriate treatment” in this context means to obtain more efficient treatment of CML, in particular in patients with lower response to imatinib. Lower response to imatinib or its pharmaceutical salts means a SHP1 level lower than 3. “Appropriate treatment” includes increasing therapeutic amount of imatinib or a pharmaceutically acceptable salt thereof, additional treatment with nilotinib or dasatinib or a pharmaceutically acceptable salt thereof, or substituting imatinib treatment with treatment with nilotinib or dasatinib or a pharmaceutically acceptable salt thereof.

A further aspect of present invention relates to a diagnostic kit comprising
a) means for determining the mRNA level of SHP1 and/or SHP2 from a sample;
b) means for determining the mRNA level of ABL;
c) means for normalizing SHP1 and/or SHP2 mRNA to ABL.

Preferably, SHP1 level is determined with such ex vivo method. Determination and normalizing is preferably performed with the methods as described in the experimental section below. Preferably, the blood sample is a peripheral blood sample.

A further aspect of present invention relates to the use of imatinib, nilotinib, and/or dasatinib, or a pharmaceutically acceptable salt thereof, for the treatment of a CML patient with a SHP1 level lower than about 3.

A further aspect of present invention relates to the use of imatinib, nilotinib, and/or dasatinib, or a pharmaceutically acceptable salt thereof, for the manufacture of a medicament for the treatment of CML, wherein the SHP1 level of the patient is lower than about 3.

A further aspect of present invention relates to a method of treating CML in a warm-blooded animal comprising the steps of
(a) determining the SHP1 level before the treatment in blood of a patient suffering from CML, and
(b) administering a daily dose of Imatinib mesylate, nilotinib, or dasatinib to the patient suffering from CML showing a SHP1 level lower than about 3, wherein said daily dose of Imatinib mesylate is at least 150% of the standard dosage prescribed for CML patients.

Step b) hence comprises either increasing the therapeutic amount of imatinib or a pharmaceutically acceptable salt thereof, additional treatment with nilotinib or dasatinib or a pharmaceutically acceptable salt thereof, or substituting imatinib treatment with treatment with nilotinib or dasatinib or a pharmaceutically acceptable salt thereof. The therapeutic amount of dasatinib is in general 100 mg/day, that of nilotinib is 800 mg/day.

A further aspect of present invention relates to a method of treating chronic myeloid leukemia (CML) in a human patient comprising the steps of
(a) determining the SHP1 expression level before the treatment in blood of a patient suffering from CML, and
(b) administering a daily dose of imatinib mesylate to the patient suffering from CML showing a SHP1 expression level lower than about 3, wherein said daily dose of Imatinib mesylate is at least 150% of the standard dosage prescribed for CML patients.

A further aspect of present invention relates to a package insert for a medicament comprising imatinib, nilotinib, and/or dasatinib, or a pharmaceutically acceptable salt thereof, characterized that it contains instructions for the use for patients with an SHP1 lever lower than about 3.

In another aspect, the present invention pertains to a method of treating CML in a warm-blooded animal comprising the steps of increasing the daily dose of Imatinib mesylate, nilotinib, or dasatinib to the patient suffering from CML showing a lower SHP2.

The information regarding standard dosage prescribed for CML patients can be normally obtained from the label contained in the drug package.

In a preferred embodiment, said daily dose of Imatinib mesylate, nilotinib or dasatinib is 150%, 200%, 250% or 300% of the standard dosage prescribed for CML patients.

For example, in the case when standard dosage prescribed for CML patients is 400 mg, the daily dose to be administered to patients having lower SHP1 is between about 800 and 1200 mg of Imatinib mesylate, e.g. 600 mg/day, 800 mg/day, 1000 mg/day or 1200 mg/day.

Preferred amounts of imatinib mesylate in case of a SHP1 level lower than 3 are 600 mg/day to 1200 mg/day. Further preferred lower limits are 650 mg/day, 700 mg/day, 750 mg/day, 800 mg/day and 850 mg/day. Further preferred upper limits are 1150 mg/day, 1100 mg/day, 1050 mg/day, 1000 mg/day, 950 mg/day and 900 mg/day. It is to be understood that each combination of upper and lower limits are comprised in present invention.

In an embodiment, in step (b) a daily dose of Imatinib mesylate is administered orally,

Imatinib is generically and specifically disclosed in the patent applications U.S. Pat. No. 5,521,184, in particular in Example 21, the subject-matter of which is hereby incorporated into the present application by reference. Imatinib can also be prepared in accordance with the processes disclosed in WO03/066613.

For the purpose of the present invention, Imatinib is preferably applied in the form of its mono-mesylate salt, imatinib mono-mesylate can be prepared in accordance with the processes disclosed in U.S. Pat. No. 6,894,051 the subject-matter of which is hereby incorporated into the present application by reference. Comprised are likewise the corresponding polymorphs, e.g. crystal modifications, which are disclosed therein.

Imatinib mono-mesylate can be administered in dosage forms as described in U.S. Pat. No. 5,521,184, U.S. Pat. No. 6,894,051 or U.S. 2005-0267125.

Nilotinib is for instance disclosed in WO2004005281, example 92, the subject-matter of which is hereby incorporated into the present application by reference.

Dasatinib is for instance disclosed in WO 00/62778.

Detection of SHP1 and/or SHP2 level:

The collecting of a blood sample from CML patients can be accomplished by standard procedures being state of the art. The Q-PCR is performed as below:

One microgram of total RNA extracted from the patient samples or cell lines, was prewarmed for 10 min at 70° C.; the RNA solution was then incubated for 10 min at 25° C., 45 min at 42° C. and 3 min at 99° C. in a 20 μL reaction mixture containing 10 mM Tris-HCl (pH 8.3), 50 mM KCl, 5.5 mM MgCl_{2,} 1 mM of each deoxyribonucleotide, 20 U of RNAsin (Pharmacia, Upsala, Sweden), 25 mM random examers (Pharmacia), 10 mM of DTT (Pharmacia), and 100 U of MoMLV reverse transcriptase (invitrogen Ltd). PCR amplification of SHP-1 and SHP-2 encoding cDNAs were separately carried out in a reaction mixture consisting of 1×Master Mix (Applied BioSystem, Foster City, Calif. USA), 300 nM of the appropriate primer pair and 200 nM of the appropriate probe in a final volume of 25 μL using the following time/temperature profile: 95° C., 15 s, and 60° C., 1 min, for 50 cycles. All amplification reactions were carried out in triplicate. The primers and probes sequences were as follows; SHP1: 139 bp; Forward: CGAGGTGTCCACGGTAGCTT, Reverse: CCCCTCCATACAGGTCATAGAAAT, Probe: Fam-TGACCCATATTCGGATCCAGAACTCAGG-Tamra; SHP2; 89 bp; Forward: GCGACAACTGCACGGATCT, Reverse: CAGCGTCACAGCCCCTAAG, Probe: Fam-CTCGCACTGGGAATCCCCTCCAT-Tamra. ABL: 123 bp; Forward: TGGAGATAACACTCTAAGCATAACTAAAGGT, Reverse: GATGTAGTTGCTTGGGACCCA, Probe: Fam-CCATTTTTGGTTTGGGCTTCACACCATT-Tamra. ABL was used as an internal control. SHP1 and SHP2 mRNA was normalized to ABL, All reaction were performed using an ABI-7900 sequence detector (Applied BioSystem).

### Clinical Studies

In one study we have evaluated the expression levels of two SHP-constitutive non receptor protein tyrosine phosphatases, the SHP-1 and SHP-2, in leukemia cells obtained from newly diagnosed CML patients enrolled into the TOPS (Tyrosine kinase inhibitor Optimization and Selectivity) trial. TOPS is a prospective, open-label, randomized (2:1) Phase III trial that compared Ima 800 mg/d to 400 mg/d in CP-CML, The end point of the trial is the rate of major molecular response (MMR), which has been indicated by several reports as an indicator that predicts a benefit for progression free survival (PFS), Our hypothesis was that differential levels of SHP 1 and SHP2 are associated with patients achieving MMR, when compared to those who did not achieve MMR at 12 months. The initial results obtained from 48 newly diagnosed CML patients enrolled into the TOPS trial, have shown that the expression levels of both SHP1 and SHP2, as assessed by QPCR in peripheral blood of these patients and expressed as ratio to ABL, are significantly different between those patients who do and do not achieved MMR by 12 months. Specifically, SHP1/Abl % was 7.4±3.8 vs 5.0±3.2, (p=0.017) and SHP2/ABL % was 0.19±0.15 vs 0.10±0.12 (p=0.017).

In this study, we have first used, as model system, a couple of Ima-sensitive (KCL22s) and Ima-resistant (KCL22r) KCL22 cell lines. In these cells, Ima resistance is independent by the oncogenic Bcr/Abl activity. We have found a very low level of Shp1 (both mRNA and protein), a protein with a tumour suppressor activity, in the KCL22r resistant cells, when compared to KCL22s sensitive cells. We have also shown the down-regulation of this gene to be related to the methylation level of SHP1 promoter, indeed, 5-Azacytidine (5-AC) treatment, along with demethylation of the promoter region, re-induced expression of Shp1 in KCL22r. That treatment also re-established the Ima sensitivity, i.e. Ima growth inhibition, in these cells. At molecular level, the restored Ima sensitivity was associated to a significant reduction of phosphorylation of both STAT3 and ERK1/2, To better understand the functional role of Shp1, we carried out mass spectrometry to search for Shp1-binding proteins, and found that Shp1 interacts in these cells with Shp2, a protein phosphatase well known as positive regulator of oncogenic pathways, including the Ras/MAPK pathway. Gain-of-function mutations have been described in various hemopoietic neoplasias including Juvenile Chronic Myelomonocytic Leukemia. In Ph+ cells, oncogenic Bcr/Abl protein activates Shp2 through Gab2, an adaptor protein that, once phosphorylated is able to bind SH2 domain of Shp2. Through complex interactions that may involve the two carboxy-terminal tyrosine residues (542 and 580) Shp2 is also a signal transducer of growth factor receptor. We hypothesized that, Shp1, through dephosphorylation, might modulate the activity of Shp2 and constitute an important mechanism of Ima resistance. Knock-down of Shp1 in KCL22s cell line resulted in complete phosphorylation of Shp2 both 542 and 580 tyrosine residues and in its reduced sensitivity to the drug, thus supporting the role of this protein in Ima sensitivity. On the other hand, knock-down of Shp2 in KCL22r, that shows low Shp1 level, resulted in growth inhibition, restored Ima sensitivity and is associated to a significant reduction of phosphorylation of both STAT3 (80%) and ERK1/2 (70%). The data on primary cells support the role of Shp1 in Ima resistance in patients.

Indeed, we analyzed bone marrow samples of 60 CML patients classified, according to the ELN definitions, as optimal (n=35), suboptimal (n=17) Ima responder, and primary (n=5) or secondary resistant (n=3) to Ima. The levels of Shp1 mRNA were significantly reduced in resistant patients [ratio of SHP1/ABL 3.2±1.04, (mean±SD),*p21 0.05] when compared to the suboptimal (3.8±1.54) and optimal responders (5.8±1.77). Moreover, the Shp1 decrease was observed in CD34+0 cells isolated from 6 resistant patients in comparison to 6 optimal responders. In conclusion, our study suggests that an aberrant balance between the Shp1 and 2 levels play a role in the Bcr-Abl independent resistance to Ima through activation of Ras/MAPK pathway and that lower levels of Shp1 are associated with non responsive patients.

In this study we investigated the predictive role of the levels of expression of two SHP-constitutive non receptor protein tyrosine phosphatase, the SHP-1 and SHP-2, in leukemia cells obtained from 48 newly diagnosed CML patients enrolled into the TOPS (Tyrosine kinase inhibitor Optimization and Selectivity) trial. TOPS is a prospective, open-label, randomized (2:1) Phase III trial that compared Ima 800 mg/d to 400 mg/d in CP-CML. The findings end point of the trial is the rate of major molecular response (MMR) indicated by several reports as a parameter that predict a benefit for progression free survival (PFS). Results indicate that the mRNA levels of both SHP1 and SHP2 assayed by QPCR in peripheral blood of newly diagnosed the patients and expressed as ratio to ABL, are significantly different between those patients who do and do not achieved MMR by 12 months (7.4±3.8 vs 5.0±3.2, p=0.017 for SHP1/Abl % and 0.19±0.15 vs 0.10±0.12, p=0.017 for SHP2/ABL %).

To further explore the role of SHP1 as a determinant of imatinib sensitivity we evaluated the expression of SHP1 in 93 newly-diagnosed CML patients enrolled into the TOPS-Tyrosine kinase inhibitor Optimization and Selectivity trial—(Cortes et at, EHA 2008). The results of this study indicate that the mRNA levels of SHP1, as assessed by QPCR in peripheral blood of patients at the time of enrolment, are significantly different between patients who do or don't achieve MMR by 12 months (7.9±4.0 vs. 5.9±3.4; p=0.01). Logistic regression was used to estimate regression coefficients and corresponding odds ratio using MMR by 12 months as outcome variable in our model. Since the 25^{th} and 75^{th} percentiles of SHP1 were 4.3 and 8.4, respectively (resulting in an interquartile range of 4.1), statistical analysis shown that a value of 4.1 or more in SHP1 is associated with almost 2-fold odds of achieving MMR by 12 months (OR=1.92; 95% Cl=1.12, 3.29; p=0.018). Moreover, in a contingency table chi-square analysis shown a high risk of not achieving MMR at 12 month in those patients with either low SHP1 expression and high Sokal score, when compared with patients with high-intermediate SHP1 expression and low-intermediate Sokal score (p=0.0068). In conclusion, these results suggest that, measuring expression levels of SHP1 could be of value in assessing newly diagnosed CP-CML patients and estimating treatment response, which could help optimizing Gleevec treatment, or recommending patients to more potent TKIs.

In conclusion, our results indicate, that the levels of expression of SHP1 and SHP2 are useful predictors of MMR in newly diagnosed CP-CML patients.

1-2. (canceled)

3. An ex vivo method for determining the SHP1 and/or SHP2 level, comprising the steps of
a) determining the mRNA level of SHP1 and/or SHP2 from a sample;
b) determining the mRNA level of ABL;
c) normalising SHP1 and/or SHP2 mRNA to ABL.

4. Use of the method according to claim 3 for screening CML patterns to determine appropriate treatment with imatinib, nilotinib, and/or dasatinib, or a pharmaceutical acceptable salt thereof.

5. A diagnostic kit comprising
a) means for determining the mRNA level of SHP 1 and/or SHP2 from a sample;
b) means for determining the mRNA level of ABL;
c) means for normalizing SHP1 and/or SHP2 mRNA to ABL.

6-8. (canceled)

9. A method of treating chronic myeloid leukemia (CML) In a human patient comprising the steps of
(a) determining the SHP1 expression level before the treatments blood of a patient suffering from CML, and
(b) administering a daily dose of Imatinib mesylate to the patient suffering from CML showing a SHP1 expression level lower than about 3, wherein said daily dose of Imatinib mesylate is at feast 150% of the standard dosage prescribed for CML patients.

10. (canceled)
